SEQUENCE LISTING (15 sequences)

SEQ ID NO: 1 - RNA, 34 nt, Artificial sequence
  Synthetic oligonucleotide
  misc_feature (1)..(20): n at positions 1 to 20 is a, g, c or u.
nnnnnnnnnnnnnnnnnnnnguuuuagagcuaga

SEQ ID NO: 2 - RNA, 65 nt, Artificial sequence
  Synthetic oligonucleotide
aauagcaaguuaaaauaaggcuaguccguuaucaacuugaaaaaguggcaccgagucggugcuuu

SEQ ID NO: 3 - RNA, 34 nt, Artificial sequence
  Synthetic oligonucleotide
  misc_feature (34)..(34): A at position 34 is linked to C6-NH2 moiety.
gcugaauuacucacgccccaguuuuagagcuaga

SEQ ID NO: 4 - RNA, 34 nt, Artificial sequence; identical to SEQ ID NO: 3
  Synthetic oligonucleotide
  misc_feature (34)..(34): A at position 34 is linked to C6-N3 moiety.

SEQ ID NO: 5 - RNA, 34 nt, Artificial sequence; identical to SEQ ID NO: 3
  Synthetic oligonucleotide
  misc_feature (34)..(34): A at position 34 is linked to 2′-O-[2-(2-azidoethyl)methylcarbamate]).

SEQ ID NO: 6 - RNA, 34 nt, Artificial sequence
  Synthetic oligonucleotide
  misc_feature (34)..(34): A at position 34 is linked to C6-NH2.
guguauuuugaccuacgaauguuuuagagcuaga

SEQ ID NO: 7 - RNA, 34 nt, Artificial sequence; identical to SEQ ID NO: 6
  Synthetic oligonucleotide
  misc_feature (34)..(34): A at position 34 is linked to C6-N3.

SEQ ID NO: 8 - RNA, 34 nt, Artificial sequence; identical to SEQ ID NO: 6
  Synthetic oligonucleotide
  misc_feature (34)..(34): A at position 34 is linked to 2′-O-[2-(2-azidoethyl)methylcarbamate].

SEQ ID NO: 9 - RNA, 65 nt, Artificial sequence; identical to SEQ ID NO: 2
  Synthetic oligonucleotide
  misc_feature (1)..(1): A at position 1 is linked to hexyne.

SEQ ID NO: 10 - RNA, 99 nt, Artificial sequence
  Synthetic oligonucleotide
  misc_feature (34)..(35): A at position 34 is joined via a linker (L) to A at position 35.
guguauuuugaccuacgaauguuuuagagcuagaaauagcaaguuaaaauaaggcuaguccguuaucaacuugaaaaaguggcaccgagucggugcuuu

SEQ ID NO: 11 - RNA, 99 nt, Artificial sequence; identical to SEQ ID NO: 10
  Synthetic oligonucleotide

SEQ ID NO: 12 - RNA, 81 nt, Artificial sequence
  Synthetic oligonucleotide
guguauuuugaccuacgaauguuuuagagcuagaaauagcaaguuaaaauaaggcuaguccguuaucaacuugaaaaagug

SEQ ID NO: 13 - RNA, 42 nt, Artificial sequence
  Synthetic oligonucleotide
guguauuuugaccuacgaauguuuuagagcuaugcuguuuug

SEQ ID NO: 14 - RNA, 74 nt, Artificial sequence
  Synthetic oligonucleotide
aacagcauagcaaguuaaaauaaggcuaguccguuaucaacuugaaaaaguggcaccgagucggugcuuuuuuu

SEQ ID NO: 15 - RNA, 20 nt, Artificial sequence
  Synthetic oligonucleotide
guguauuuugaccuacgaau

(19) United States
(12) Patent Application Publication
(10) Pub. No.: US 2018/0142236 A1
(43) Pub. Date: May 24, 2018

(54) SYNTHETIC SINGLE GUIDE RNA FOR CAS9-MEDIATED GENE EDITING

(71) Applicant: GE HEALTHCARE DHARMACON, INC., Wauwatosa, WI (US)

(72) Inventors: Kaizhang He, Lafayette, CO (US); Emily Marie Anderson, Lafayette, CO (US); Michael Oren Delaney, Lafayette, CO (US)

(21) Appl. No.: 15/571,532

(22) PCT Filed: Apr. 7, 2016
(86) PCT No.: PCT/US16/26444

§ 371 Date: Nov. 3, 2017

Related U.S. Application Data
(60) Provisional application No. 62/162,209, filed on May 15, 2015.

(51) Int. Cl. C12N 15/11; C12N 9/22

(52) U.S. Cl. CPC C12N 15/111; C12N 9/22; C40B 40/06; C12N 2310/318; C12N 2310/3519; C12N 2310/20

(57) ABSTRACT

The present invention provides synthetic single guide RNAs that comprise two separate functional sequences (commonly known as crRNA and tracrRNA) connected by a linker. These synthetic single guide RNA molecules are useful in gene editing when used with RNA-guided endonucleases such as cas9 in eukaryotic cells. The availability of the synthetic single guide RNAs makes the screening for gene editing in high-through-put format simple and convenient.

### FIELD OF THE INVENTION

The present invention relates to the field of gene editing.

### BACKGROUND OF THE INVENTION

For many years, researchers have looked to the use of oligonucleotides to control activity within a cell. Among the processes that have been explored are those that rely on antisense technologies and RNA interference (“RNAi”) technologies. Each of these technologies makes use of the ability of an oligonucleotide to target a region or regions of one or more other nucleic acids based on a degree of complementarity of the relevant nucleotide sequences.

One area that has recently been explored in connection with controlling the activity of DNA is the use of the CRISPR-Cas system. The CRISPR-Cas system makes use of proteins that occur naturally in about 40% -60% of bacteria and about 90% of archaea. Naturally occurring CRISPR proteins, in combination with certain types of non-translated RNA, have been shown to confer resistance in these prokaryotes to foreign DNA. Within these prokaryotes, CRISPR loci are composed of cas genes that are arranged in operons and a CRISPR array that consists of unique genome-targeting sequences that are called spacers and are interspersed with identical repeats.

Recently, researchers reported developing a method for controlling gene expression using Cas9, which is an RNA-guided DNA endonuclease from a type II CRISPR system. Typically, they described success in gene editing by using the Cas9 protein derived from S. pyogenes when it is co-expressed with a guide RNA (“gRNA”). In this context, the gRNA is a chimeric molecule of two separate RNA molecules, i.e., a DNA targeting sequence (crRNA) fused with a non-targeting transactivating sequence (tracrRNA). Alternatively, one can achieve efficient gene editing by employing two separate synthetic RNAs, crRNA and tracrRNA, in Cas9 expressing cells or by co-transfecting into cells with a Cas9 expression vector, Cas9 protein or Cas9 mRNA. Unfortunately, due to its size (˜116 nts) and low yield, chemical synthesis of a single guide RNA molecule has not been possible to be of practical use. The present invention solves this problem.

### SUMMARY OF THE INVENTION

The present invention is directed to various chemically synthesized single guide RNA molecules that are useful for modulating and/or modifying DNA. Through the use of various technologies disclosed herein, including oligonucleotides and oligonucleotide:protein complexes, one can efficiently and effectively control activity in a cell or cells within an organism.

According to the first embodiment, the present invention provides a synthetic single guide RNA comprising a first oligonucleotide comprising a sequence complementary to a sequence in a target DNA, a second oligonucleotide comprising a sequence that interacts with a site-directed modifying polypeptide, wherein the first oligonucleotide and the second oligonucleotide are joined via a non-phosphodiester covalent linkage. The first oligonucleotide is typically about 25-60 nucleotides in length, the second oligonucleotide is typically about 40-100 nucleotides in length. Any one of the nucleotides therein can be chemically modified, for example, 2′-modification.

Examples of the covalent linkage include but are not limited to: those having a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C-C bond forming groups such as Diels-Alder cyclo-addition pairs or ring-closing metathesis pairs, and Michael reaction pairs.

The site-directed modifying polypeptides are RNA-guided DNA endonucleases having an RNA binding portion that interacts with the synthetic single guide RNA and an activity portion that exhibits site-directed enzymatic activity, e.g. double stranded DNA cleavage. One example of the site-directed modifying polypeptide is Cas9 derived from a type II CRISPR system and the Cas9 polypeptide can be a wild type protein as it exists in nature, a mutant Cas9 (e.g. point mutation, deletion mutation or truncated), or a chimeric polypeptide that is fused with another functional peptide. The target DNA is any DNA, preferably eukaryotic DNA, more preferably mammalian DNA, most preferably human DNA. The target sequence may be a coding region of template strand of DNA, a coding region of a non-template strand of DNA, or a non-coding region such as a promoter region of a template strand of DNA or a promoter region of a non-template strand of DNA, an enhancer region of a template strand or non-template strand or an insulator region of a template strand or non-template strand. The target sequence can also be non-coding sequences encoding long non-coding RNAs (lncRNAs).

According to a second embodiment, the present invention provides a composition comprising the synthetic single guide RNA of the first embodiment and a site-directed modifying polypeptide or a polynucleotide encoding the same. One example of the site-directed modifying polypeptide is Cas9 derived from a type II CRISPR system and the Cas9 polypeptide can be a wild type protein as it exists in nature, a mutant Cas9 (e.g. point mutation, deletion mutation or truncated) or a chimeric polypeptide that is fused with another functional peptide. In certain embodiments the polynucleotide encoding the modifying polypeptide is cas9 mRNA that has been transcribed in vitro. In other embodiments, the polynucleotide encoding the modifying polypeptide is a plasmid DNA expressing the modifying protein or a viral particle (e.g. lentiviral particle) expressing the modifying polypeptide.

According to a third embodiment, the present invention provides a method of site-specific modification of a target DNA, said method comprising introducing into a cell or contacting a cell with the synthetic single guide RNA of the first embodiment and a site-directed modifying polypeptide or a polynucleotide encoding the same. One example of the site-directed modifying polypeptide is Cas9 derived from a type II CRISPR system and the Cas9 polypeptide can be a wild type protein as it exists in nature, a mutant Cas9 (e.g. point mutation, deletion mutation or truncated) or a chimeric polypeptide that is fused with another functional peptide. In certain embodiments the polynucleotide encoding the modifying polypeptide is Cas9 mRNA that has been transcribed in vitro. In other embodiments, the polynucleotide encoding the modifying polypeptide is a plasmid DNA expressing the modifying protein or a viral particle (e.g. lentiviral particle) expressing the modifying polypeptide.

In addition, the present invention provides a library of the synthetic single guide RNAs of the first embodiment. The library may consist of at least 10, 30, 50, 75, or at least 100 RNA molecules, at least 500, or at least 1000 RNA molecules, each of which targets a different sequence in a target DNA. In this instance the target DNA can be the same gene targeted by multiple sgRNAs or multiple genes targeted by e.g. each sgRNA targeting different gene. The library can also be in the form of a pool of at least 2 synthetic single guide RNAs or an individual RNA in each well in a multi-well format.

Various embodiments of the present invention provide one or both of increased gene editing efficiency, specificity, and ease of use.

### BRIEF DESCRIPTION OF THE FIGURES

FIG. 1 shows the steps of preparing 3′-azido-adenosine polystyrene support.

FIG. 2 shows the steps of preparing 5′-hexyne phosphoramidite.

FIG. 3 exemplifies the synthetic steps for the single guide RNA of the invention.

FIG. 4 shows the results of the T7E1 mismatch detection assay demonstrating that the synthetic single guide RNA of 99 nucleotides that has been ligated by a linker (lanes D and E) can cleave the human PPIB gene at a comparable level of efficiency compared to the cleavage of the same target gene carried out by the use of two separate molecules complexed as crRNA:tracrRNA (lane C); lane A: synthetic 99mer not conjugated; lane B: synthetic 81mer not conjugated.

### DETAILED DESCRIPTION

The present invention provides oligonucleotide molecules, complexes, systems, other compositions and methods for creating and using these molecules, complexes, systems, and other compositions in order to modulate and/or to modify endogenous regions of eukaryotic DNA and/or chromatin and/or other moieties associated with DNA and/or chromatin. Through the various embodiments of the present invention, one can effectively and efficiently alter activity in vitro and in vivo with the desired level of specificity.

### Definitions

Unless otherwise stated or implicit from context, the following words, phrases, abbreviations and acronyms have the meanings provided below:

The abbreviation “Cas” refers to a CRISPR-associated moiety, e.g., a protein such as Cas9 from a Type II system or derivatives thereof. Cas9 proteins constitute a family of enzymes (i.e., RNA guided DNA endonucleases) that in naturally occurring instances rely on a base-paired structure to be formed between an activating tracrRNA and a targeting crRNA in order to cleave double-stranded DNA. In a naturally occurring tracrRNA:crRNA secondary structure, there is base-pairing between the 3′-terminal 22-nucleotides of the crRNA and a segment near the 5′ end of the mature tracrRNA. This interaction creates a structure in which e.g. the 5′ terminal 20 nucleotides of the crRNA can vary in different crRNAs and are available for binding to target DNA when the crRNA is associated with a Cas protein.

The abbreviation “CRISPR” refers to Clustered Regularly Interspaced Short Palindromic Repeats. CRISPRs are also known as SPIDRs—Spacer Interspersed Direct Repeats and constitute a family of DNA loci. These loci typically consist of short and highly conserved DNA repeats, e.g., 24-50 base pairs that are repeated 1-40 times and that are at least partially palindromic. The repeated sequences are usually species specific and are interspaced by variable sequences of constant length, e.g., 20-58 base pairs. A CRISPR locus may also encode one or more proteins and one or more RNAs that are not translated into proteins. Thus, a “CRISPR-Cas” system is a system that is the same as or is derived from bacteria or archaea and that contains at least one Cas protein that is encoded or derived by a CRISPR locus. For example, the S. pyogenes SF370 type II CRISPR locus consists of four genes, including a gene for the Cas9 nuclease, as well as two non-coding RNAs: tracrRNA and a pre-crRNA array that contains nuclease guide sequences (spacers) interspaced by identical repeats (DRs).

The abbreviation “crRNA” refers to a CRISPR RNA. crRNAs may be obtained from a CRISPR array that may be transcribed constitutively as a single long RNA that is then processed at specific sites. A crRNA can also be chemically synthesized. A crRNA molecule comprises the DNA targeting segment and a stretch of nucleotides that forms one half of the imperfect dsRNA duplex of the protein binding segment of the DNA targeting RNA.

The terms, “guide RNA” and “single guide RNA” are used interchangeably herein. When the guideRNA (gRNA) is made by chemical means, it's referred to as “synthetic single guide RNA” or “synthetic sgRNA”. The guide RNA refers to a polynucleotide sequence comprising two different functional sequences, crRNA and tracrRNA, in their native size or form or modified. The gRNA can be expressed using an expression vector or chemically synthesized. The synthetic sgRNA can comprise a ribonucleotide or analog thereof or a modified form thereof, or an analog of a modified form, or non-natural nucleosides. The synthetic single guide RNA can also contain modified backbones or non-natural internucleoside linkages.

The term, “linker”, as used herein, refers to a chemical entity that joins at least two separate oligonucleotide molecules. In some embodiments, the first oligonucleotide and the second oligonucleotide are covalently ligated via the 3′ end of the first oligonucleotide and the 5′ end of the second oligonucleotide. Alternatively, the first and the second oligonucleotides can be covalently ligated via the 5′ end of the first oligonucleotide and the 3′ end of the second oligonucleotide.

The term “nucleotide” includes a ribonucleotide or a deoxyribonucleotide. In some embodiments, each nucleotide is a ribonucleotide or analog thereof or a modified form thereof, or an analog of a modified form. Nucleotides include species that comprise purine nucleobases, e.g., adenine, hypoxanthine, guanine, and their derivatives and analogs, as well as pyrimidines, e.g., cytosine, uracil, thymine, and their derivatives and analogs.

Examples of modified bases include but are not limited to nucleotides such as the following nucleotides: adenine, guanine, cytosine, thymine, uracil, xanthine, inosine, and queuosine, wherein there has been a modification by the replacement or addition of one or more atoms or groups. The replacement or addition may cause the nucleotide to be alkylated, halogenated, thiolated, aminated, amidated, or acetylated at one or more positions.

More specific examples of modified bases include, but are not limited to, 5-propynyluridine, 5-propynylcytidine, 6-methyladenine, 6-methylguanine, N, N,-dimethyladenine, 2-propyladenine, 2-propylguanine, 2-aminoadenine, 1-methylinosine, 3-methyluridine, 5-methylcytidine, 5-methyluridine, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deazaadenosine, 6-azouridine, 6-azocytidine, 6-azothymidine, 5-methyl-2-thiouridine, and other thio bases such as 2-thiouridine and 4-thiouridine and 2-thiocytidine, dihydrouridine, pseudouridine, queuosine, archaeosine, naphthyl and substituted naphthyl groups, any O-and N-alkylated purines and pyrimidines such as N6-methyladenosine, 5-methylcarbonylmethyluridine, uridine 5-oxyacetic acid, pyridine-4-one, and pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

Modified nucleotides also include those nucleotides that are modified with respect to the sugar moiety, as well as nucleotides having sugars or analogs thereof that are not ribosyl. For example, the sugar moieties may be, or be based on mannoses, arabinoses, glucopyranoses, galactopyranoses, 4′-thioribose, and other sugars, heterocycles, or carbocycles. One type of modification of the sugar moiety is a modification of the 2′ position. Examples of 2′-ribose modifications include but are not limited to replacing the —OH group with moieties such as —H (hydrogen), —F, —NH₃, —OCH₃ and other O-alkyl moieties (e.g., —OC₂H₅, and —OC₃H₇), alkenyl moieties, alkynyl moieties and orthoester moieties.

The term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes. Perfect complementarity or 100% complementarity refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other. The synthetic single guide RNA disclosed herein comprises a nucleotide sequence, for example 10-20 nucleotides in length, which is complementary to a sequence in the target DNA. However this complementarity does not have to be contiguous as long as the synthetic single guide RNA is capable of being used to modify a sequence in the target DNA in a sequence dependent manner

The phrase, “site-directed modifying polypeptide” means a polypeptide or protein that binds RNA and is targeted to a specific DNA sequence. The site-directed modifying polypeptide that can be used in the present invention is RNA-guided DNA endonucleases which are targeted to a specific DNA sequence by the synthetic single guide RNA molecule to which it is bound and thus cleave double-stranded target DNA. Preferred RNA-guided DNA endonucleases for the invention are Cas9 proteins from a Type II CRISPR-Cas system or derivatives thereof, either a wild type protein as it exists in nature, a mutant Cas9 including a truncated Cas9 protein or a chimeric cas9 polypeptide with a distinct functional domain (e.g. transcription activator) fused to a native Cas9 protein or a fragment of Cas9 protein.

The acronym “PAM” refers to a protospacer adjacent motif. A PAM is typically 3-5 nucleotides in length and located adjacent to protospacers in CRISPR genetic sequences, downstream or 3′ of the nontargeted strand. PAM sequences and positions can vary according to the CRISPR-Cas system type. For example, in the S. pyogenes Type II system, the PAM has a NGG consensus sequence that contains two G:C base pairs and occurs one base pair downstream of the protospacer-derived sequence within the target DNA. The PAM sequence is present on the non-complementary strand of the target DNA (protospacer), and the reverse complement of the PAM is located 5′ of the target DNA sequence. The PAM sequence may be specific to the system, e.g., the system from which the site-directed modifying protein is derived.

The term, “chimeric” as used herein as applied to nucleic acid or polypeptide refers to two components that are defined by structures derived from different sources. For example, where chimeric is used in the context of a chimeric polypeptide, the chimeric polypeptide includes amino acid sequences that are derived from two different polypeptides. A chimeric polypeptide may contain either modified or naturally occurring polypeptide sequences. Examples of chimeric site-directed modifying polypeptides that can be used with the synthetic single guide RNA of the invention include but are not limited to the polypeptide having enzymatic activity that modifies target DNA, for example, methyltransferase activity, demethylase activity, DNA repair activity, polymerase activity, recombinase activity, helicase activity, integrase activity.

The terms, “peptide”, “polypeptide” or “protein” are used interchangeably herein and refer to a polymeric form of amino acids of any length, which can include coded or non-coded amino acids, chemically or biochemically modified or derived amino acids, and polypeptides having modified peptide backbones.

Whenever a range is given in the specification, for example, a temperature range, a time range, a percent sequence identity, a sequence complementarity range, a length range, or a composition or concentration range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of ” and “consisting of ” may be replaced with either of the other two terms. The disclosure illustratively described herein suitably may be practiced in the absence of any element or elements, limitation or limitations which is not specifically disclosed herein.

### Synthetic Single Guide RNA

The present invention provides synthetic single guide RNAs that are useful in modifying a specific locus in a target DNA when used with a site-directed modifying polypeptide such as Cas9. The synthetic single guide RNAs are comprised of two oligonucleotides covalently linked. The first oligonucleotide (known as crRNA) contains a sequence that is complementary to a nucleotide sequence in a target DNA and a sequence that associates with tracrRNA. The second oligonucleotide (also known as tracrcRNA) is comprised of a nucleotide sequence that interacts with a site-directed modifying polypeptide (e.g. Cas9) and a sequence that associates with the first oligonucleotide. A synthetic single guide RNA and a site-directed modifying polypeptide form a complex which targets and cleaves a target DNA at a specific sequence determined by a complementary sequence in the first oligonucleotide.

The synthetic single guide RNA of the invention has several advantages compared to the guide RNA made by other means, e.g. vector expressed or transcribed in vitro; i) it is simple to design, make, and test their functionality, ii) the nucleotides can be chemically modified to enhance stability and specificity if desired, and iii) it is amenable to construct a large number of single gRNAs for high-through-put (HTP) screening purposes. Furthermore, the use of conjugation chemistry to link the two separate oligonucleotides circumvents the problem of low yield of chemical synthesis of longer RNAs.

The synthetic single guide RNA of the present invention is typically about 65-160 nucleotides in length, e.g. about 66-120 nucleotides, about 70-110 nucleotides, about 81-99 nucleotides in length. In one embodiment, the first oligonucleotide is about 25-60 nucleotides in length, and the second oligonucleotide is about 40-100 nucleotides in length. In some embodiments, the first oligonucleotide is about 30 -55 nucleotides in length, about 35-50 nucleotides in length, or about 40-45 nucleotides in length. Within the first oligonucleotide, there is a region or sequence (“targeting sequence”) that is complementary to a target sequence. In some embodiments, the targeting sequence is 18, 19, or 20 nucleotides long. It is understood that the targeting sequence needs not be 100% complementary to the target sequence. A targeting sequence can comprise at least 70%, at least 80%, at least 90%, at least 95%, or 100% complementary to a target sequence. In some embodiments, the second oligonucleotide is about 50-90 nucleotides in length, about 60-80 nucleotides in length or about 70-75 nucleotides in length. In certain cases, the first oligonucleotide can comprise a targeting sequence of 18 nucleotides in length and the tracr associating sequence of at least 7 nucleotides, at least 10 nucleotides, at least 15 nucleotides or at least 22 nucleotides in length. In some cases, the first nucleotide is about 42 nucleotides long and the second nucleotide is about 74 nucleotides long. In certain examples, the first nucleotide is about 34 nucleotides long and the second nucleotide is about 65 nucleotides long. In yet another example, the first nucleotide is 34 nucleotides long and the second nucleotide is 47 nucleotides long.

In certain embodiments, at least one nucleotide of the first oligonucleotide and the second oligonucleotide may be chemically modified. For example, any of the nucleotides in the first and second oligonucleotides may comprise a 2′-modification. In other embodiments, the first nucleotide, the second nucleotide and the last nucleotide of the synthetic sgRNA may be chemically modified singly or in combination. In some embodiments, each nucleotide other than the first nucleotide, the second nucleotide, and the last nucleotide contains a 2′OH group on its ribose sugar. In some instances, either the first oligonucleotide or the second oligonucleotide or both the first and the second oligonucleotides may contain modified oligonucleotides.

The synthetic sgRNA of the invention can comprise any corresponding crRNA and tracrRNA pair as they exist in nature. The crRNA and tracRNA sequences are known in the art from several type II CRISPR-Cas9 systems (WO2013/176772).

### Conjugation of First Oligonucleotide and Second Oligonucleotide

The synthetic single guide RNA of the invention is of typically about 65 to 160 nucleotides in length and can be represented by a formula:

A-L-B

Where A is the first oligonucleotide of about 25-60 nucleotides long, L is a flexible linker group, and B is the second oligonucleotide of about 40-100 nucleotides long.

In order to prepare a single guide RNA of the invention, two separate oligonucleotides (first and second oligonucleotides) are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some cases, the first oligonucleotide or second oligonucleotide contains an appropriate functional group for ligation with the second or the first oligonucleotide when the synthesis is complete. If, however, the first or second oligonucleotide does not contain an appropriate functional group for ligation, it can be functionalized using the standard protocol known in the art (Hermanson, G. T., Bioconjugate Techniques, Academic Press (2013)).

Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once the first oligonucleotide and the second oligonucleotide are functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C-C bond forming groups such as Diels-Alder cyclo-addition pairs or ring-closing metathesis pairs, and Michael reaction pairs.

The present invention is exemplified using the type II CRISPR-Cas9 system derived from S. pyogenes SF370. In this system, the crRNA is 42 nucleotides long and the tracrRNA is 74 nucleotides long in its naturally occurring state. It has been shown that there is base-pairing between the 3′ terminal 22 nucleotides of the crRNA and a segment near the 5′ end of the tracrRNA, which enables a complex formation with Cas9 and leads to cleave double stranded DNA in a sequence specific manner.

One example of the synthetic single guide RNA disclosed herein is 99 nucleotides long: the first oligonucleotide of 34mer conjugated with the second oligonucleotide of 65mer (see Table 1, ODN-6).

The nucleotide sequence of the base-pairing region of the first oligonucleotide (34mer) is shown below (from S. pyogenes SF370):

5′-N₂₀-GUUUUAGAGCUAGA-3′ (SEQ ID NO:1) where N₂₀ denotes the sequence complementary to a target sequence.

The nucleotide sequence of the second oligonucleotide (65mer) is shown below (from S. pyogenes SF370):

(SEQ ID NO: 2)
5'-AAUAGCAAGUUAAAAUAAGGCUAGUCCGUUAUCAACUUGAAAAAGUG
GCACCGAGUCGGUGCUUU-3'

Although the examples disclosed are based on the crRNA, tracrRNA and the Cas9 polypeptide derived from S. pyrogenes, one can adapt the sequences of crRNA, tracrRNA and cas9 polypeptide from any type II CRISPR-Cas9 systems to practice the current invention. The known type II CRISPR-Cas9 systems include but are not limited to those found in S. thermophilis, S. aureus, S. mutants, L. innocua, N. meningitides, P. multocida, M. mobile. Accordingly, one can utilize the crRNA and tracrRNA sequences from these systems and design and synthesize the sgRNAs as described herein to use with corresponding Cas9 polypeptide, functional homolog or chimeric Cas9 to achieve modification of a target DNA. See WO 2013/176772 for details including nucleotide sequences for crRNAs and corresponding tracrRNAs, and Cas9 proteins.

### The Synthetic Single Guide RNA and Protein Complex

When the first oligonucleotide and second oligonucleotide of the synthetic single guide RNA form an appropriate secondary structure, regardless of the type of modifications, the synthetic sgRNA is capable of associating with a site-directed modifying polypeptide. The site-directed modifying protein comprises an RNA association region and an activity region. The RNA association region is capable of associating with the sgRNA at or near the double-stranded region, and the activity region is capable of causing an action with respect to the target or with respect to molecules or moieties associated with the target.

In some embodiments, the modifying protein is a naturally occurring Cas9 that has endonuclease activity. In other embodiments, the modifying protein is a non-naturally occurring Cas9 that lacks endonuclease activity. For example, it may be a Cas9 protein derived from S. pyrogenes that contains inactivating mutations of the RuvC1 and HNH nuclease domains (e.g. D10A and H841A, WO 2013/141680) or lacks these domains, but optionally is engineered to have a different activity domain or an inactive activity domain.

In some embodiments, the modifying protein is capable of recognizing a protospacer adjacent moiety (PAM) of a target DNA and/or binding directly to a DNA element. A DNA element may be a single-stranded or a double-stranded stretch of DNA nucleotides or chromatin or the proteins within chromatin e.g., histones. In some embodiments, site specific activity, e.g., cleavage of the target occurs at locations that are determined by both: (1) base-pairing complementarity between the targeting region of the first oligonucleotide and the target; and (ii) the PAM sequence in the target.

Alternatively or additionally, the modifying protein has a helicase activity. The helicase activity permits the protein to unwind the DNA target sequence that is specified by the targeting sequence of the first oligonucleotide. When the DNA is unwound, the targeting sequence can base pair with the DNA target.

### Methods

The oligonucleotides and complexes of the present invention may be used in vitro or in vivo to cause a change in a cell or in an organism. For example, according to the present invention, one may introduce into a cell, a single strand oligonucleotide, i.e., synthetic single guide RNA, that comprises a first oligonucleotide and a second oligonucleotide linked as described above.

One may also introduce a site-directed modifying protein. The modifying protein may be introduced from outside the cell before, after or at the same time that one introduces the single strand synthetic sgRNA that comprises a first oligonucleotide segment attached to a second oligonucleotide segment by a linker. The components may be introduced as a complex or they may form a complex within the cell.

Introduction may be passively or through a vehicle and the synthetic gRNA and the modifying protein may be present in a buffer at the time of introduction. Thus, in some embodiments the modifying protein or a synthetic gRNA or vector coding the modifying protein may be part of a kit. Alternatively, a messenger RNA encoding a modifying protein can also be used with a synthetic gRNA for gene editing.

Alternatively, the modifying protein may already be present within the cell or it may be generated from within the cell from a vector. The vector may, for example, be a recombinant expression vector that comprises a DNA polypeptide that codes for the modifying protein. In some embodiments, when a vector is used, it contains an inducible promoter.

In another embodiment, one may introduce into a cell, a synthetic sgRNA that comprises a chemically modified oligonucleotide as described above. As with other methods, one may also introduce a modifying protein. The modifying protein may be introduced from outside the cell before, after or at the same time that one introduces the guide RNA. Alternatively, the modifying protein may already be present within the cell or it may be generated from within the cell from a vector. In some embodiments, when a vector is used, it contains an inducible promoter.

Once all of the components are within the cell or nucleus and the complex is formed, a targeting region of the first oligonucleotide or targeting sequence that is located at or near the 5′ end of the first oligonucleotide directs the complex to a target by the complementarity of the targeting region to the target. The activity region of the complex then acts upon the target sequence, expression of the target sequence or a moiety within the proximity of the target sequence.

If one or more components are to be generated by an inducible promoter, then the molecule that induces the promoter should be introduced prior to commencing or while carrying out the method.

The methods may cause the increase or decrease in expression or expression rate of a protein, or cause the increase or decrease in transcription rate. By way of a non-limiting example, the methods may cause site directed modification of target DNA. By way of further examples, the methods may cause changes in DNA or associated proteins through one or more of the following activity regions of a modifying protein: nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, polymerase activity, ligase activity, helicase activity, glycolase activity, acetyltransferase activity, deacyltransferase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. For example, if the activity site is a nuclease, when the method is carried out, the modifying protein introduces a double strand break in the target DNA. The activity region may be part of or derived from a naturally occurring modifying protein, or it may be fused to a naturally occurring protein or part of a chimeric protein that is not naturally occurring.

In some embodiments, the methods are carried out under conditions that allow for nonhomologous end joining or homology directed repair. Furthermore, in some embodiments, the method comprises contacting target DNA with a donor polypeptide. The donor polypeptide may then integrate into the target DNA. For details, see Maggio et al. Trends Biotechnol 2015 May 33(5) 280-294 and Chen et al Nature Methods 2011 Sept: 8(9) 753-757.

### Systems

The present invention also provides systems. The systems contain each of the components of the complex or a combination of a vector from which any one or more of the components of the complex can be generated and one or more oligonucleotides, e.g., an oligonucleotide that contains the crRNA and tracrRNA as a single RNA molecule.

In one embodiment, the present invention provides a system for altering a moiety in a cell or expression of a moiety in a cell. This system comprises a vector expressing a site-directed modifying protein and a synthetic single guide RNA. The cell may be or become a genetically modified cell. In some cases, the cell is or is derived from a cell selected from the group consisting of an archaeal cell, a bacterial cell, a eukaryotic cell, a eukaryotic single cell organism, a somatic cell, a germ cell, a stem cell, a plant cell, an algae cell, an animal cell, an invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell and a human cell.

The vector, when present, is capable of expressing a modifying protein through transcription into an RNA sequence that is transcribed into a protein. The modifying protein comprises an oligonucleotide association region and an activity region as described above. Optionally, the vector may contain an inducible promoter. When vectors are used, the vector may, for example, be a plasmid DNA or a viral particle. In one embodiment, a Cas9 protein is expressed from an anhydrotetracycline (aTC)-inducible promoter on a plasmid that contains a ColE1 replication origin. In another example, a doxycycline inducible expression system is used.

Within the vector that codes for the modifying protein, there may be a sequence that codes for a fluorescent protein and/or a selection marker protein such as puromycin or blasticidin. The sequence that codes for the fluorescent protein or a marker protein may be under the control of the same promoter that codes for the modifying protein or it may be on the same vector but under the control of a different promoter. Alternatively, it may be present on a different vector under the control of a separate promoter. When there is a separate promoter that is responsible for the fluorescent protein or a selection marker protein, that promoter may be inducible by the same or different molecule or stimulus that is capable of inducing transcription of the sequence that codes for the modifying protein.

### EXAMPLES

The embodiments described herein are for illustrative purposes. Unless otherwise specified or apparent from content, any feature recited in connection with one embodiment may be used in connection with any other embodiment.

### Example 1

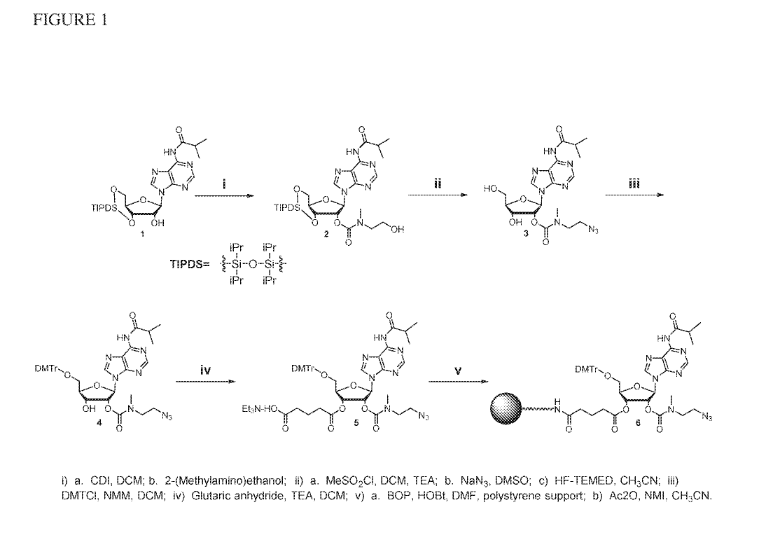

### Preparation of 3′-azidoadenosine polystyrene Support (FIG. 1)

### 1

N⁶-Isobutyryl 2′O-[2(2 hydroxyethyl)methylcarbamate]-3′,5′-O-(tetraisopropyl-disiloxane-1,3-diyl)adenosine (2)

To a solution of compound 1 (10.0 g, 17.2 mmol) in 170 mL of dichloromethane (DCM) was added CDI (1,1′-carbonyldiimidazole) (2.9 g, 18.1 mmol). After 18 h of stirring, 2-(methylamino)ethanol (5.2 g, 68.8 mmol) was added. The reaction was stopped after 1.5 h and evaporated to dryness. The crude material was purified on a Biotage Isolera using a 100 g Ultra cartridge with an ethyl acetate:MeOH gradient (0→10%) to give 2 (10.8 g, 93%) as a white foam. Compound 2 was analyzed by RP-HPLC: 10.54 min, 99.4%. ¹H NMR (CDCl₃, 300 mHz) δ8.65 (s, 1 H), 8.63 (s, 1 H), 8.10 (s, 1 H), 6.04 (d, J=8.8 Hz, 1 H), 5.64 (d, J=5.3 Hz, 1 H), 5.15 (m, 1 H), 4.16-3.98 (m, 4 H), 3.76 (m, 2 H), 3.56-3.15 (m, 3 H), 3.05 and 2.96 (each as s, 3 H), 2.86 (s, 1 H), 2.59 (m, 1 H), 1.27 (d, J=6.8 Hz, 6 H), 1.08-1.01 (m, 28 H).

### N⁶-Isobutyryl-2′-O-[2-(2-azidoethyl)methylcarbamate] adenosine (3)

To a solution of compound 2 (6.0 g, 8.8 mmol) in 44 mL of DCM was added triethylamine (2.7 g, 26.4 mmol). The solution was cooled on an ice bath and then methanesulfonyl chloride (1.2 g, 10.6 mmol) was added slowly over 5 minutes. After stirring for 30 minutes, the reaction was diluted with 100 mL of DCM and transferred to a separatory funnel. The organic phase was washed successively with 10% citric acid (2×50 mL), water (1×50 mL), and saturated NaCl (1×50 mL). The organic phase was passed over a pad of Na₂SO₄ and concentrated down to leave N⁶-Isobutyryl-2′-O-[2-(2-methanesulfonate-oxyethyl)methylcarbamate]-3′,5′-O-(tetraisopropyl-disiloxane-1,3-diyl)adenosine as a white foam, which was analyzed by RP-HPLC: 10.97 min, 96.7%. ¹H NMR (CDCl₃, 300 mHz) δ8.62 (s, 1 H), 8.58 (s, 1 H), 8.11 (s, 1 H), 6.06 (s, 1 H), 5.64, (d, J=4.7 Hz, 1 H), 5.15 (m, 1 H), 4.39-4.26 (m, 2 H), 4.15-3.99 (m, 3 H), 3.85-3.68 (m, 1 H), 3.60-3.41 (m, 1 H), 3.23-3.17 (m, 1 H), 3.07 and 3.02 (each as s, 3 H), 1.27 (d, J=6.8 Hz, 6 H), 1.08-1.00 (m, 28 H).

This material was directly dissolved in 20 mL of dimethylsulfoxide (DMSO) and to this solution was added sodium azide (1.9 g, 29.2 mmol). The suspension was then heated to 60° C. for 10 h and then diluted with 100 mL of water. The reaction mixture was extracted with Et₂O (3×100 mL). The combined ether extracts were washed with water (1×50 mL), and then with saturated NaCl (1×50 mL). The solution was dried over Na₂SO₄ and then concentrated down to give N⁶-isobutyryl-2′-O-[2(2-azidoethyl)methylcarbamate]-3′,5′-O-(tetraisopropyl-disiloxane-1,3-diyl)adenosine as a white foam (5.5 g, 89%), which was analyzed byRP-HPLC: 11.88 min, 94.1%. ¹H NMR (CDCl₃, 300 mHz) δ8.64 (s, 1 H), 8.61 (s, 1 H), 6.04 (d, J=3.1 Hz, 1 H), 5.65 (d, J=5.3 Hz, 1 H), 5.15 (m, 1 H), 4.16-3.99 (m, 3 H), 3.54-3.37 (m, 3 H), 3.27-3.18 (m, 1 H), 3.05 and 2.97 (each as s, 3 H), 1.27 (d, J=6.8 Hz, 6 H), 1.08-1.00 (m, 28 H).

This material was taken onto the desilylation step without any additional purification. To a solution of TEMED (4.50 g, 39.0 mmol) in 31 mL of CH₃CN at 0° C. was added 48% HF (1.0 mL, 27.3 mmol) dropwise. This solution was stirred for 10 min and added to N⁶-isobutyryl-2′-O-[2-(2-azidoethyl)methylcarbamate]-3′,5′-O-(tetraisopropyldisiloxane-1,3-diyl)adenosine(5.5 g, 7.8 mmol) in a separate flask. The reaction was stirred for 2 h and concentrated to dryness. The crude material was purified on a Biotage Isolera using a 50 g Ultra cartridge with a 85:15 ethyl acetate:hexanes (0.1% TEMED) to 6% MeOH in ethyl acetate (0.1% TEMED) gradient to afford compound 3 as a white foam (3.3 g, 81% from 2). Compound 3 was analyzed by RP-HPLC: 4.78 min, 96.1%. ¹H NMR (CDCl₃, 300 mHz) δ9.08 (bs, 1 H), 8.63 (s, 1 H), 8.21 (d, J=3.6 Hz, 1 H), 6.18 (d, J=6.1 Hz, 1 H), 5.66 and 5.59 (each as m, 1 H), 4.76 (m, 1 H), 4.27 (m, 1 H), 3.00-2.94 (m, 1 H), 3.81-7.77 (m, 1 H), 3.57-3.38 (m, 1 H), 3.31-3.20 (m, 4 H), 2.94 and 2.84 (each as s, 3 H), 1.24 (d, J=6.8 Hz, 6 H).

### 5′-O-Dimethoxytrityl-N⁶-isobutyryl-[2′-O-[2(2 azidoethyl)methylcarbamate]-adenosine (4)

To a solution of compound 3 (3.3 g, 7.1 mmol) in 70 mL of DCM was added N-methylmorpholine (2.3 g, 21.3 mmol). DMT-chloride (2.63 g, 7.8 mmol) was titrated into the reaction in 0.2 equivalent increments allowing the red color to dissipate between additions. The addition of 1.1 equivalents of DMT-chloride took about 20 min and the reaction was complete. The reaction was diluted with 50 mL of DCM and washed with saturated NaCl (1×50 mL). The solution was dried over Na₂SO₄ and concentrated. The crude material was purified on a Biotage Isolera using a 50 g Ultra cartridge with a DCM-acetone gradient (0→30%) to afford 4 as a white foam (4.7 g, 86%). Compound 4 was analyzed by RP-HPLC: 8.64 min, 98.9%. ¹H NMR (CDCl₃, 300 mHz) δ8.63 (s, 1 H), 8.57 (s, 1 H), 8.5 (s, 1 H), 7.40-7.16 (m, 9 H), 6.76 (d, J=8.6 Hz, 4 H), 6.32-6.28 (m, 1 H), 5.75 and 5.67 (each as m, 1 H), 4.81-4.75 (m, 1 H), 4.25 (m, 1 H), 3.75 (s, 6 H), 3.69-3.59 (m, 5 H), 3.51-3.35 (3 H), 2.98 and 2.71 (each as s, 3 H), 1.26 (d, J=6.8 Hz, 6 H).

### 5′-O-Dimethoxytrityl-N⁶-isobutyryl 2′-O[2(2 azidoethyl)methylcarbamate]-adenosine-3′-O-gluturate triethylammonium salt (5)

To a solution of compound 4 (4.7 g, 6.1 mmol) in 50 mL of DCM was added N-methylimidazole (0.25 g, 3.1 mmol) and triethylamine (3.7 g, 36.6 mmol). Glutaric anhydride (1.1 g, 9.8 mmol) was added to the reaction mixture and the solution was stirred for 18 hours at room temperature. The reaction was diluted with 50 mL of DCM and washed with saturated 5% (w/v) KH₂PO₄ (1×40 mL). The organic phase was dried over Na₂SO₄ and concentrated. The crude material was purified on a Biotage Isolera using a 50 g Ultra cartridge with a DCM-MeOH gradient (0-13%) with 2% TEA present as a cosolvent to afford compound 5 as a white foam (4.9 g, 82%). Compound 5 was analyzed by RP-HPLC: 7.47 min, 95.2%. ¹H NMR (CDCl₃, 300 mHz) δ8.90 (bs, 1 H), 8.64 (s, 1 H), 8.16 (d, J=6.3 Hz, 1 H), 7.39 (d, J=7.3 Hz, 2 H), 7.30-7.15 (m, 7 H), 6.78 (d, J=8.5 Hz, 4 H), 6.34 (m, 1 H), 5.97 (m, 1 H), 5.69 (m, 1 H), 4.33 (m, 1 H), 3.74 (s, 6 H), 3.39-3.30 (m, 6 H), 3.12-3.98 (m, 3 H), 2.89 and 2.87 (each as s, 3 H), 2.49-2.30 (m, 5 H), 1.97-1.91 (m, 2 H), 1.25 (m, 10 H).

### Derivatization of Aminomethylated Polystyrene Support (6):

To a solution of compound 5 (0.044 g, 0.045 mmol) in 13 mL of DMF was added triethylamine (0.009 g, 0.09 mmol), BOP (0.022 g, 0.05 mmol), and HOBt (0.007 g, 0.054 mmol). The solution was allowed to activate for 5 minutes and then 10.8 mL (1.3 equivalents) of this solution was added to a suspension of aminomethylated polystyrene support (5 g) in 30 mL of DMF. The suspension was shaken for 1 hour and then the loading was monitored by DMT assay. Loading was determined to be 6.4 umol/g. The suspension was then filtered in a coarse fritted funnel and washed with acetone (300 mL). The dried support was transferred to a flask and dried in a vacuum desiccator. After drying overnight, the loaded support was capped with a solution of 10% acetic anhydride and 10% N-methylimidazole in CH₃CN. The suspension was shaken for 3 h, and then filtered through a coarse fritted funnel. The solid material remaining was washed with acetone (300 mL) and then dried in a vacuum desiccator until ready for use.

### Example 2

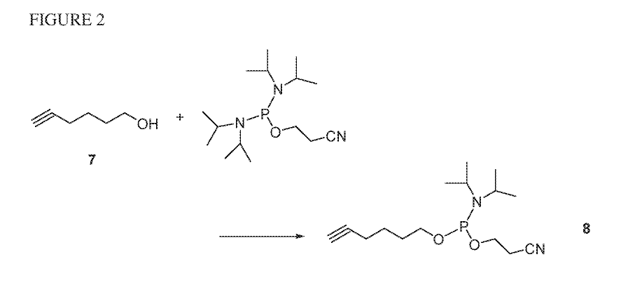

### Preparation of 5′-hexyne phosphoramidite (8) (FIG. 2)

Compound 7 (hex-5-yn-1-ol, 1.4 mL) was dissolved with 10 mL DCM in a flask and N,N-diisopropylamine (1.82 mL) was added to the solution. In a separate flask under anhydrous conditions, the phosphinylating reagent bis-(N,N-diisopropylamino)-cyanoethylphosphine (1.5 equiv per equiv 7) was diluted with DCM (2 mL per mmol phosphine) and a solution of 0.45 M 1H-tetrazole in MeCN (0.5 equiv tetrazole per equiv 7) was added and shaken for 5 min. Next, the solution of activated phosphinylating reagent was added to the well-stirred solution of compound 7 at room temperature and stirred at room temperature until the reaction is complete by TLC analysis. To quench the excess phosphine ethanol was added and the reaction mixture was stirred for additional 30 minutes and dried on the rotary evaporator. The product was purified on silica gel to give 0.8 g of phosphoramidite 8. ³¹P NMR (CDCl₃, 121.5 mHz) δ147.0 (s).

### Example 3

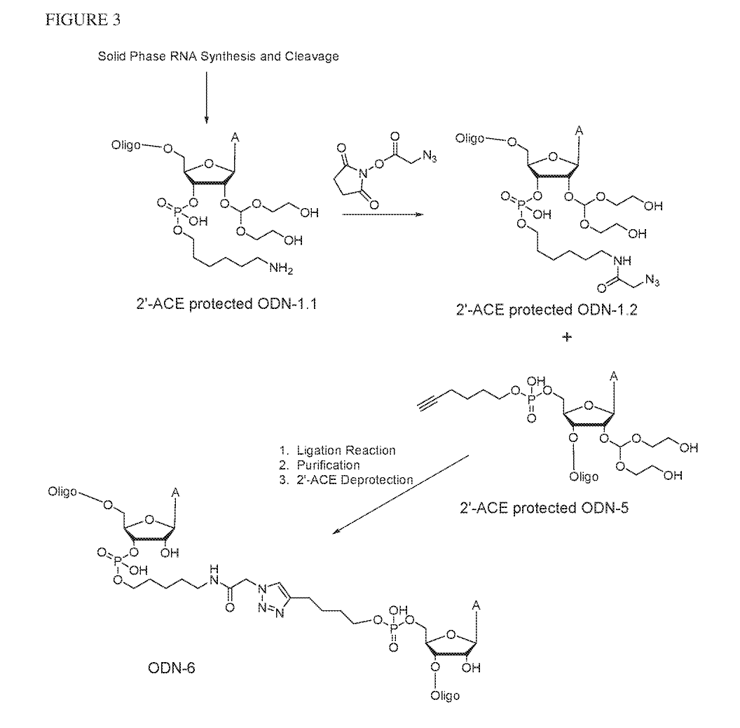

### Conjugated Oligonucleotide Synthesis (Table 1 and FIG. 3)

2′-ACE protected RNA oligonucleotides (ODN-1.1, ODN-2, ODN-3.1, ODN-4, ODN-5, ODN-7, and ODN-8) were chemically synthesized on a MerMade synthesizer (Bioautomation Corporation, Irving, Tex..) using polystyrene solid supports and 2′-bis(acetoxyethoxy)-methyl ether (2′-ACE) phosphoramidites. For ODN-2 and ODN-4, aminomethylated polystyrene support 6 (see Example 1) was employed. For ODN-5, 5′-hexyne phosphoramidite 8 was used. After completion of synthesis cycles, the oligonucleotide on the support was treated with Na₂S₂ solution at room temperature followed by washing with water. The oligonucleotide was cleaved from the support with 40% of aqueous N-methylamine (NMA) and then heated at 55° C. followed by lyophilization to dryness. The crude RNA was desalted, purified by HPLC, and the identity of the purified sample was confirmed by UPLC and ESI-MS.

ODN-1.2 and ODN-3.2: Azidoacetic acid NHS ester (Click Chemistry Tools) in DMF was added post-synthetically to the freeze dried 3′-aminoalkyl-modified oligonucleotide (2′-ACE protected ODN-1.1 or ODN-3.1) in Na₂CO₃/NaHCO₃ buffer. The azide-labeled oligonucleotide was desalted and purified by reverse-phase HPLC.

Ligation reaction in the presence of Cu(I): 5′-Hexyne-modified oligonucleotide (2′-ACE protected ODN-5) (50 nmol) was dissolved in water and 2M TEAA buffer (pH 7.0). 3′-Azide-labeled oligonucleotide (2′-ACE protected ODN-3.2) (75 nmol, 10 mM stock solution in DMSO) was then added. A stock 5 mM solution of ascorbic acid (175 uL) was added followed by degassing the solution with argon. A pre-made solution (10 mM in 55% DMSO) of Cu(II)-TBTA (87 uL) was added to the mixture. The mixture was allowed to react at room temperature overnight. Using the same ligation conditions, ODN-2 or ODN-4 can be conjugated with ODN-5 to make the synthetic sgRNAs targeting two different target genes.

The conjugated oligonucleotide (2′-ACE protected ODN-6) was precipitated with acetone. The pellet was washed with acetone, dried, and purified by reverse-phase HPLC. 2′-ACE groups were removed by adding Dharmacon's 2′-deprotection buffer (100 mM acetic acid-TEMED, pH 3.4-3.8) with 30 minute incubation at room temperature. The conjugated RNA oligonucleotide (ODN-6) was desalted by ethanol precipitation and ready for use.

TABLE 1
Oligonucleotides synthesized

| | | Nu- | SEQ |
|---|---|---|---|
| | | cleotide | ID |
| # | Nucleotide Sequences | Length | No: |
| ODN- | 5'-GCUGAAUUACUCACGCCCCAGUUUU | 34 | 3 |
| 1.1 | AGAGCUAGA-C6-NH₂-3' | | |
| ODN- | 5'-GCUGAAUUACUCACGCCCCAGUUUU | 34 | 4 |
| 1.2 | AGAGCUAGA-C6-N₃-3' | | |
| ODN-2 | 5'-GCUGAAUUACUCACGCCCCAGUUUU | 34 | 5 |
| | AGAGCUAGA(2'-O-[2-(2- | | |
| | azidoethyl) | | |
| | methylcarbamate])-3' | | |
| ODN- | 5'-GUGUAUUUUGACCUACGAAUGUUUU | 34 | 6 |
| 3.1 | AGAGCUAGA-C6-NH₂-3' | | |
| ODN- | 5'-GUGUAUUUUGACCUACGAAUGUUUU | 34 | 7 |
| 3.2 | AGAGCUAGA-C6-N₃-3' | | |
| ODN-4 | 5'-GUGUAUUUUGACCUACGAAUGUUUU | 34 | 8 |
| | AGAGCUAGA(2'-O-[2-(2- | | |
| | azidoethyl) | | |
| | methylcarbamate])-3' | | |
| ODN-5 | 5'-Hexyne- | 65 | 9 |
| | AAUAGCAAGUUAAAAUAAGGCUAGUCCG | | |
| | UUAUCAACUUGAAAAAGUGGCACCGAGU | | |
| | CGGUGCUUU-3' | | |
| ODN-6 | 5'-GUGUAUUUUGACCUACGAAUGUUUU | 99 | 10 |
| | AGAGCUAGA-L-AAUAGCAAGUUAAAAU | | |
| | AAGGCUAGUCCGUUAUCAACUUGAAAAA | | |
| | GUGGCACCGAGUCGGUGCUUU-3' | | |
| ODN-7 | 5'-GUGUAUUUUGACCUACGAAUGUUUU | 99 | 11 |
| | AGAGCUAGAAAUAGCAAGUUAAAAUAAG | | |
| | GCUAGUCCGUUAUCAACUUGAAAAAGUG | | |
| | GCACCGAGUCGGUGCUUU-3' | | |
| ODN-8 | 5'-GUGUAUUUUGACCUACGAAUGUUU | 81 | 12 |
| | UAGAGCUAGAAAUAGCAAGUUAAAAUA | | |
| | AGGCUAGUCCGUUAUCAACUUGAAAAA | | |
| | GUG-3' | | |

### Where L is:

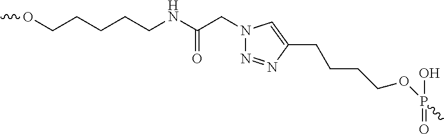

### Example 4

### Gene Editing Activity of Synthetic Single Guide RNA

HEK293T cells stably expressing S. pyrogenes Cas9 protein were seeded in a 96-well plate at a density of 10,000 cells per well. The following day crRNA (42mer, 5′-GUGUAUUUUGACCUACGAAUGUUUUAGAGCUAUGCUGUUUUG-3′: SEQ ID NO: 13 and tracrRNA (74mer, 5′-AACAGCAUAGCAAGUUAAAAUAAGGCUAGUCCGUUAUCAACUUGAAAAAG UGGCACCGAGUCGGUGCUUUUUUU-3′: SEQ ID NO: 14) or three synthetic sgRNAs, 81mer (ODN-8), 99mer (ODN-7), and conjugated 99mer (ODN-6) were individually resuspended in 10 mM Tris-HCl (pH7.5), 100 mM NaCl, and 1 mM EDTA to 100 μM. crRNA and tracrRNA were added together to form a complex and the RNA was further diluted to 5 μM using sterile 1× X siRNA Buffer (Dharmacon, B-002000-UB-100). A final concentration of 25 nM crRNA:tracrRNA complex (25 nM of each crRNA and tracrRNA) or synthetic sgRNA was used for transfection. The cells were transfected with 25 nM crRNA:tracrRNA complex or synthetic sgRNA using DharmaFECT 1 Transfection Reagent (Dharmacon, #T-2001-03).

Genomic DNA was isolated 72 hours post-transfection by direct lysis of the cells in Phusion HF buffer (Thermo Scientific, #F-518L), proteinase K and RNase A for 20 minutes at 56° C. followed by heat inactivation at 96° C. for 5 minutes. PCR was performed with primers flanking the cleavage sites in the target gene PPIB. 500 ng of PCR products were treated with T7 endonuclease I (T7EI; NEB, #M0302L) for 25 minutes at 37° C. and the samples were separated on a 2% agarose gel. Percent editing (indel formation) in each sample was calculated using ImageJ.

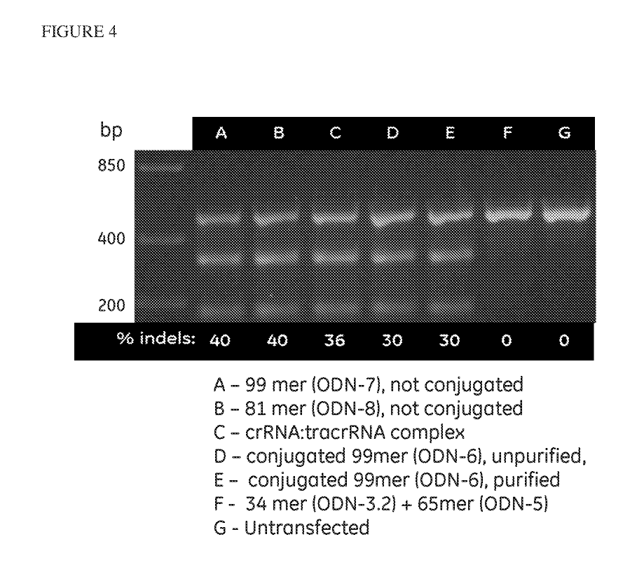

As shown in FIG. 4, the synthetic sgRNA that has been conjugated (99mer labeled as ODN-6 in Table 1) is active for gene editing (see lanes D and E) as demonstrated by the T7E1 mismatch detection assay. Also shown in FIG. 4 are several control RNA molecules; lane A is a synthetic RNA of 99mer (not conjugated) and lane B is a synthetic RNA of 81mer (not conjugated), both of which are active in gene editing. The 81mer has the same crRNA (34 nucleotides) as the 99mer but the sequence is truncated from the 3′ end of the tracrRNA (5-GUGUAUUUUGACCUACGAAUGUUUUAGAGCUAGAAAUAGCAAGUUAAAAU AAGGCUAGUCCGUUAUCAACUUGAAAAAGUG-3′: SEQ ID NO:12). Both an unpurified batch of the conjugated material (land D) and a purified batch (lane E) produce significant editing compared to the crRNA:tracrRNA complex (lane C). The precursors of the conjugation reaction do not produce editing, as demonstrated in lane F. The 20mer targeting sequence (5′-GUGUAUUUUGACCUACGAAU-3′; SEQ ID NO: 15) is designed to target the beginning of exon 2 of the human PPIB gene, chr15:64,454,334-64,454,353.

All references cited in the present application are incorporated in their entirety herein by reference to the extent not inconsistent herewith.

1. A synthetic single guide RNA comprising:
(i) a first oligonucleotide comprising a sequence that is complementary to a sequence in a target DNA;
(ii) a second oligonucleotide comprising a sequence that interacts with a site-directed modifying polypeptide,
wherein the first oligonucleotide and the second oligonucleotide are joined via a non-phosphodiester covalent linkage.

2. The synthetic single guide RNA of claim 1, wherein the first oligonucleotide is about 25-60 nucleotides in length and the second oligonucleotide is about 40-100 nucleotides in length.

3. The synthetic single guide RNA of claim 1, wherein the covalent linkage comprises a chemical moiety selected from the group consisting of carbamate, ether, ester, amide, imine, amidine, aminotrizine, hydrozone, disulfide, thioether, thioester, phosphorothioate, phosphorodithioate, sulfonamide, sulfonate, fulfone, sulfoxide, urea, thiourea, hydrazide, oxime, triazole, photolabile linkage, C-C bond forming group such as Diels-Alder cyclo-addition pair or ring-closing metathesis pair, and Michael reaction pair.

4. The synthetic single guide RNA of claim 1, wherein the site-directed modifying polypeptide is a Cas9 polypeptide.

5. The synthetic single guide RNA of claim 1, wherein the Cas9 polypeptide is derived from S. pyrogenes.

6. The synthetic single guide RNA of claim 1, wherein the Cas9 polypeptide is derived from S. thermophilis.

7. The synthetic single guide RNA of claim 1, wherein at least one nucleotide of the first oligonucleotide or second oligonucleotide is chemically modified.

8. The synthetic single guide RNA of claim 7, wherein at least one nucleotide that is chemically modified comprises a 2′-modification.

9. The synthetic single guide RNA of claim 1, wherein the site-directed modifying polypeptide is a chimeric site-directed modifying polypeptide.

10. The synthetic single guide RNA of claim 1, wherein the target DNA is mammalian DNA.

11. The synthetic single guide RNA of claim 10, wherein the mammalian DNA is human DNA.

12. The synthetic single guide RNA of claim 1, wherein the Cas9 polypeptide comprises at least one mutation such that the enzymatic activity is reduced or eliminated.

13. A composition comprising:
(i) a synthetic single guide RNA comprising:
(a) a first oligonucleotide comprising a nucleotide sequence that is complementary to a sequence in a target DNA;
(b) a second oligonucleotide comprising a sequence that interacts with a site-directed modifying polypeptide, wherein the first oligonucleotide and the second oligonucleotide are joined via a non-phosphodiester covalent linkage;
(ii) a site-directed modifying polypeptide, or a polynucleotide encoding the same, the site-directed modifying polypeptide comprising:
(a) an RNA binding portion that interacts with the synthetic single guide RNA; and
(b) an activity portion that exhibits site-directed enzymatic activity, wherein the site of enzymatic activity is determined by the nucleotide sequence of the synthetic single guide RNA.

14. The composition of claim 13, wherein the synthetic single guide RNA is about 65-160 nucleotides in length.

15. The composition of claim 13, wherein the covalent linkage comprises a chemical moiety selected from the group consisting of carbamate, ether, ester, amide, imine, amidine, aminotrizine, hydrozone, disulfide, thioether, thioester, phosphorothioate, phosphorodithioate, sulfonamide, sulfonate, fulfone, sulfoxide, urea, thiourea, hydrazide, oxime, triazole, photolabile linkages, C-C bond forming group such as Diels-Alder cycloaddition pair or ring-closing metathesis pair, and Michael reaction pair.

16. The composition of claim 13, wherein the site-directed modifying polypeptide is a Cas9 polypeptide.

17. The composition of claim 13, wherein the synthetic single guide RNA contains at least one chemically modified nucleotide.

18. The composition of claim 17, wherein the chemically modified nucleotide comprises a 2′-modification.

19. (canceled)

20. (canceled)

21. (canceled)

22. (canceled)

23. (canceled)

24. (canceled)

25. (canceled)

26. A library of the synthetic single guide RNAs of claim 1 wherein the library comprises at least 10 RNA molecules.
